(19) United States
(12) Patent Application Publication
(10) Pub. No.: US 2005/0026984 A1
(43) Pub. Date: Feb. 3, 2005

(54) Substituted thieno [2,3-c] pyrazoles and their use as medicinal products

(75) Inventors: Antony Bigot, Massy (FR); Francois Clerc, Antony (FR); Gilles Doerflinger, Les Ulis (FR); Serge Mignani, Chatenay-Malabry (FR); Herve Minoux, Thiais (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 10/900,549

(22) Filed: Jul. 28, 2004

Related U.S. Application Data
(60) Provisional application No. 60/500,614, filed on Sep. 5, 2003.

(30) Foreign Application Priority Data
Jul. 29, 2003 (FR) 0309284

(51) Int. Cl. A61K 31/4162; C07D 498/02

(52) U.S. Cl. 514/406; 548/360.5

(57) ABSTRACT

The present invention relates in particular to novel chemical compounds, particularly novel substituted thieno[2,3-c]pyrazoles, to the compositions containing them and to their use as medicinal products for treating cancers and also neurodegenerative diseases.

This application claims the benefit of U.S. Provisional Application No. 60/500,614 filed Sep. 5, 2003 and benefit of priority from French Patent Application No. 03 09284, filed Jul. 29, 2003, both of which are incorporated herein by reference in their entirety.

The present invention relates in particular to novel chemical compounds, particularly novel substituted thieno[2,3-c]pyrazoles and thieno[3,2-c]-pyrazoles, to the compositions containing them and to their use as medicinal products.

More particularly, the invention relates to specific novel indazoles exhibiting anticancer activity via modulation of the activity of proteins, in particular of kinases.

To date, most of the commercial compounds used in chemotherapy are cytotoxic agents which pose considerable problems of side effects and of tolerance in patients. These effects may be limited in so far as the medicinal products used act selectively on cancer cells, with exclusion of healthy cells. One of the solutions for limiting the adverse effects of chemotherapy may therefore consist in using medicinal products which act on metabolic pathways or elements constituting these pathways, expressed mainly in cancer cells, and which would be expressed very little or not at all in healthy cells.

Protein kinases are a family of enzymes which catalyze the phosphorylation of hydroxyl groups of specific protein residues such as tyrosine, serine or threonine residues. Such phosphorylations can widely modify the function of proteins; thus, protein kinases play an important role in regulating a large variety of cell processes, including in particular metabolism, cell proliferation, cell differentiation, cell migration or cell survival. Among the various cellular functions in which the activity of a protein kinase is involved, certain processes represent attractive targets for treating cancer-related diseases and also other diseases.

Thus, one of the objects of the present invention is to provide compositions having anticancer activity, acting in particular with respect to kinases. Among the kinases for which modulation of the activity is sought, Aurora 2 is preferred.

Many proteins involved in chromosome segregation and spindle assembly have been identified in yeast and drosophila. Disorganization of these proteins leads to non-segregation of chromosomes and to monopolar or disorganized spindles. Among these proteins, some kinases, including Aurora and Ipl1, which originate respectively from drosophila and S. cerevisiae, are necessary for chromosome segregation and separation of the centrosome. A human analogue of yeast Ipl1 has recently been cloned and characterized by various laboratories. This kinase, called Aurora2, STK15 or BTAK, belongs to the serine/threonine kinase family. Bischoff et al. have shown that Aurora2 is oncogenic and is amplified in human colorectal cancers (EMBO J, 1998, 17, 3052-3065). Examples of this have also been shown in cancers involving epithelial tumours, such as breast cancer.

Among the other kinases on which the products of the invention may act, mention may be made of FAK, KDR, Src, Tie2 and cyclin-dependent kinases

FAK is a cytoplasmic tyrosine kinase which plays an important role in transduction of the signal transmitted by integrins, a family of heterodimeric cell adhesion receptors. FAK and the integrins are colocalized in perimembrane structures called adhesion plaques. It has been shown, in many cell types, that the activation of FAK, and also phosphorylation thereof on tyrosine residues and in particular autophosphorylation thereof on tyrosine 397, are dependent of the binding of integrins to their extracellular ligands and are therefore induced during cell adhesion [Kornberg L, et al. J. Biol. Chem. 267(33): 23439-442. (1992)]. The autophosphorylation of FAK, on tyrosine 397, represents a binding site for another tyrosine kinase, Src, via its SH2 domain [Schaller et al. Mol. Cell. Biol. 14 :1680-1688. 1994; Xing et al. Mol. Cell. Biol. 5 :413-421. 1994]. Src can then phosphorylate FAK on tyrosine 925, thus recruiting the Grb2 adaptor protein and inducing, in certain cells, activation of the ras and MAP kinase pathway involved in the control of cell proliferation [Schlaepfer et al Nature; 372:786-791. 1994; Schlaepfer et al. Prog. Biophy. Mol. Biol. 71:435-478. 1999; Schlaepfer and Hunter, J. Biol. Chem. 272:13189-13195. 1997]. The activation of FAK can also induce the jun NH2-terminal kinase (JNK) signalling pathway and result in the progression of cells to the G1 phase of the cell cycle [Oktay et al., J. Cell. Biol. 145 :1461-1469. 1999]. Phosphatidylinositol-3-OH kinase (Pl3-kinase) also binds to FAK on tyrosine 397, and this interaction might be necessary for the activation of Pl3-kinase [Chen and Guan, Proc. Nat. Acad. Sci. USA. 91: 10148-10152. 1994; Ling et al. J. Cell. Biochem. 73 :533-544. 1999]. The FAK/Src complex phosphorylates various substrates, such as paxillin and p130CAS in fibroblasts [Vuori et al. Mol. Cell. Biol. 16: 2606-2613. 1996].

The results of many studies support the hypothesis that FAK inhibitors might be used in the treatment of cancer. Studies have suggested that FAK may play an important role in cell proliferation and/or survival in vitro. For example, in CHO cells, some authors have demonstrated that overexpression of p125FAK leads to an acceleration of the G1 to S transition, suggesting that p125FAK promotes cell proliferation [Zhao J.-H et aL J. Cell Biol. 143:1997-2008. 1998]. Other authors have shown that tumour cells treated with FAK antisense oligonucleotides lose their adhesion and enter into apoptosis (Xu et al, Cell Growth Differ. 4:413-418. 1996). It has also been demonstrated that FAK promotes cell migration in vitro. Thus, fibroblasts deficient for the expression of FAK (mice which are knockout for FAK) exhibit a rounded morphology and deficiencies in cell migration in response to chemotactic signals, and these deficiencies are eliminated by reexpression of FAK [D J. Sieg et al., J. Cell Science. 112:2677-91. 1999]. Overexpression of the C-terminal domain of FAK (FRNK) blocks the extension of adherent cells and decreases cell migration in vitro [Richardson A. and Parsons J. T. Nature. 380:538-540. 1996]. Overexpression of FAK in CHO or COS cells or in human astrocytoma cells promotes migration of the cells. The involvement of FAK in the promotion of cell proliferation and migration in many cell types in vitro suggests that FAK has a potential role in neoplastic processes. A recent study has effectively demonstrated an increase in the proliferation of tumour cells in vivo after induction of FAK expression in human astrocytoma cells [Cary L. A. et al. J. Cell Sci. 109:1787-94. 1996; Wang D et aL J. Cell Sci. 113:4221-4230. 2000]. In addition, immunohistochemical studies of human biopsies have demonstrated that FAK is overexpressed in prostate cancer, breast cancer, thyroid cancer, colon cancer, melanoma, brain cancer and lung cancer, the level of FAK expression being directly correlated with the tumours exhibiting the most aggressive phenotype [Weiner T M, et al. Lancet. 342(8878):1024-1025. 1993 ; Owens et al. Cancer Research. 55:2752-2755. 1995; Maung K. et al. Oncogene. 18:6824-6828. 1999; Wang D et al. J. Cell Sci. 113:4221-4230. 2000].

KDR (Kinase insert Domain Receptor), also known as VEGF-R2 (Vascular Endothelial Growth Factor Receptor 2), is expressed only in endothelial cells. This receptor binds to the angiogenic growth factor VEGF and thus acts as a mediator to a transduction signal via the activation of its intracellular kinase domain. Direct inhibition of the kinase activity of VEGF-R2 makes it possible to reduce the phenomenon of angiogenesis in the presence of exogenous VEGF (Vascular Endothelial Growth Factor) (Strawn et al., Cancer Research, 1996, vol. 56, p. 3540-3545). This process has been demonstrated in particular using VEGF-R2 mutants (Millauer et al., Cancer Research, 1996, vol. 56, p. 1615-1620). The VEGF-R2 receptor does not appear to have any function in adults other than that related to the angiogenic activity of VEGF. Consequently, a selective inhibitor of the kinase activity of VEGF-R2 should show only slight toxicity.

In addition to this central role in the dynamic angiogenic process, recent results suggest that VEGF expression contributes to the survival of tumour cells after chemotherapy and radiotherapy, underlining the potential synergy of KDR inhibitors with other agents (Lee et al. Cancer Research, 2000, vol. 60, p. 5565-5570).

It has been noted that the Src kinase, involved in many signalling cascades, is often activated or overexpressed in many types of cancer, such as colon cancer or breast cancer (Moasser M M et al. Cancer Res. 1999. 59 :6245-6152; Wiener et al. Clin. Cancer Res. 1999. 5 :2164-2170). In addition, Src appears to play a predominant role in the development of bone metastases, by virtue of its involvement in the development of bone tissue (Soriano P. et al. Cell 1991. 64:693-702; Nakagawa et al, lnt. J. Cancer 2000. 88 :384-391).

Tie-2 (TEK) is a member of a family of tyrosine kinase receptors, specific for endothelial cells. Tie2 is the first receptor possessing tyrosine kinase activity for which both the agonist (angiopoietin 1 or Ang1) which stimulates autophosphorylation of the receptor and cell signalling [S. Davis et al (1996) Cell 87, 1161-1169] and the antagonist (angiopoietin 2 or Ang2) [P. C. Maisonpierre et al. (1997) Science 277, 55-60] are known. Angiopoietin 1 can act synergistically with VEGF in the final stages of neoangiogenesis [Asahara T. Circ. Res.(1998) 233-240]. Knockout experiments and transgenic manipulations of the expression of Tie2 or of Ang1 result in animals which exhibit vascularization defects [D. J. Dumont et al (1994) Genes Dev. 8, 1897-1909 and C. Suri (1996) Cell 87, 1171-1180]. The binding of Ang1 to its receptor results in the autophosphorylation of the kinase domain of Tie2 which is essential for neovascularization and also for the recruitment and interaction of vessels with pericytes and smooth muscle cells; these phenomena contribute to the maturation and stability of the newly formed vessels [P. C. Maisonpierre et al (1997) Science 277, 55-60]. Lin et al (1997) J. Clin. Invest. 100, 8: 2072-2078 and Lin P. (1998) PNAS 95, 8829-8834, have shown an inhibition of tumour growth and vascularization and also a decrease in lung metastases during adenoviral infections or during injections of the extracellular domain of Tie-2 (Tek) in breast tumour and melanoma xenograft models.

Tie2 inhibitors can be used in situations where neovascularization takes place inappropriately (i.e. in diabetic retinopathy, chronic inflammation, psoriasis, Kaposi's sarcoma, chronic neovascularization due to macular degeneration, rheumatoid arthritis, infantile haemangioma and cancers).

Progression of the cell cycle is often controlled by cyclin-dependent kinases (CDKs) which are activated by a balance within the cyclin family, this activation ending with the phosphorylation of substrates and, finally, with cell division. In addition, endogenous inhibitors of CDKs which are activated (INK4 and KIP/CIP family) negatively regulate CDK activity. The growth of normal cells is due to a balance between the CDK activators (cyclins) and the endogenous CDK inhibitors. Aberrant expression or the activity of several components of the cell cycle has been described in several types of cancer.

Cyclin E activates the Cdk2 kinase, which then acts to phosphorylate pRb, resulting in an irreversible entry into cell division and transition to the S phase (P L Toogood, Medicinal Research Reviews (2001), 21(6); 487-498); it is also possible, according to these authors, that the CDK2 and CDK3 kinases are necessary for progression in the G1 phase and entry into S phase. During complex formation with cyclin E, they maintain the hyperphosphorylation of pRb so as to aid the progression of the G1 phase to S phase. In complexes with cyclin A, CDK2 plays a role in the inactivation of E2F and is necessary for realizing the S phase (T D. Davies et al. (2001) Structure 9, 389-3).

The CDK1/cyclin B complex regulates progression of the cell cycle between the G2 phase and the M phase. Negative regulation of the CDK/cyclin B complex prevents normal cells entering into S phase before the G2 phase has been correctly and completely realized (K. K. Roy and E. A. Sausville Current Pharmaceutical Design, 2001, 7, 1669-1687.

There is a level of regulation which exists for CDK activity. Cyclin-dependent kinase (CAK) activators have a positive regulatory action on CDKs. CAK phosphorylates CDKs on the threonine residue so as to make the target enzyme completely active.

The presence of defects in the molecules involved in the cell cycle leads to CDK activation and progression of the cycle; a standard intention is to inhibit the activity of the CDK enzymes so as to block cell growth in cancer cells.

Among the kinases on which the compounds according to the invention have an activity, mention may also be made of Glycogen Synthase Kinase-3β (GSK-3β) also known as tau 1 kinase (TPK I). It has been described as a major kinase involved in the phosphorylation of helicoidal filament bundles. GSK-3β is a proline-directed kinase which phosphorylates the tau site, for which a specific phosphorylation-dependent antibody reveals the presence of entanglements in the brain tissues of patients suffering from Alzheimer's disease. The pathogenic role of GSK-3β in neurodegeneration has also been suggested by recent in vitro tests demonstrating that GSK-3β is involved in neuron death triggered by various cytotoxic agents. The administration of selective inhibitors of GSK-3β may thus reduce the formation of helicoidal filament bundles and the gradual dysfunctioning of neurons in human tauopathies such as Alzheimer's disease. GSK-3β inhibitors thus represent a therapeutic approach for patients suffering from this disease. In addition to Alzheimer's disease, the inhibitors according to the invention can be used in the treatment or prevention of diseases resulting from an abnormal activity of this kinase, such as diabetes, Parkinson's disease, obesity, essential hypertension, atherosclerotic cardiovascular diseases, polycystic ovaries syndrome, syndrome X, immunodeficiency, cancer, and other neurodegenerative pathologies such as frontoparietal dementia, corticobasal degeneration, Pick's disease, strokes, cranial and spinal traumas and peripheral neuropathies.

The present invention relates in particular to novel chemical compounds, particularly novel substituted thieno[2,3-c]pyrazoles, to the compositions containing them and to their use as medicinal products.

More particularly, the invention relates to novel specific thieno[2,3-c]pyrazoles exhibiting anticancer activity via modulation of the activity of proteins, in particular of kinases.

The present invention relates to novel thieno[2,3-c]pyrazole derivatives. It also relates to the use of the thieno[2,3-c]pyrazoles as kinase-inhibiting agents, and more particularly as an anticancer agent. It also relates to the use of said derivatives for preparing a medicinal product intended for the treatment of humans.

Described among the prior art known to date describing thieno-[2,3-c]pyrazoles and thieno[3,2-c]pyrazoles, is most particularly patent DE 19 642 323, which describes substituted 1-(heterocyclylmethyl)-3-(aryl/heteroaryl)pyrazoles of general formula below 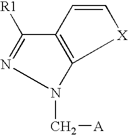
in which R1 represents an aryl or heteroaryl group, X represents O, S, NH or CH═CH and A represents an optionally substituted phenyl group. These products are used as cardiovascular agents.

Patent Application WO 03/024397 also describes a very broad heterocycle family which can include the thienopyrazoles of general formula below 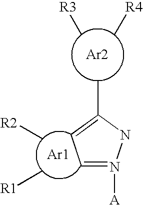
where A can denote hydrogen, Ar2 and Ar1 are, independently, phenyl or a heterocycle, and R1, R2, R3, and R4 are, independently, hydrogen, nitro, halogen, aryl, heteroaryl, OH, OR, C(O)OH, C(O)OR, C(O)SH, C(O)SR, C(O)NH2, C(O)NHR, C(O)NRR′, ROH, ROR′, RSH, RSR′, ROC(O)R′OH, NHR, NRR′, RNHR′, or RNR′R″ where R, R′ and R″ are (C1-C6)alkyl. Document WO 04/007504 relates to substituted 3-aminothieno[3,2-c]-pyrazoles and to their derivatives, which are useful for treating cancer.

Document WO 04/013146 relates to substituted 3-aminothieno[2,3-c]-pyrazoles and to their derivatives, which are useful for treating cancer, of general formula below: 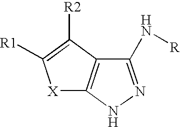
in which:

X is in particular S;

R and R1 are independently chosen from H, R′, COR′, COOR′, CONHR′. CONR′R″, SO₂R′, SO₂NHR′ and SO₂NR′R″, in which R′ and R″ are independently chosen from H, alkyl, heterocyclyl, aryl and heteroaryl;

R2 is chosen from R′, CH₂OR′ and OR′.

The compounds according to the invention:

Among the compounds corresponding to formula (I), the following compounds may be mentioned:

Formula (I) Thieno[2,3-c]pyrazoles:
3-(1H-Indol-2-yl)-1H-thieno[2,3-c]pyrazole-5-carboxylic acid benzylamide
N-[3-(1H-Indol-2-yl)-1H-thieno[2,3-c]pyrazol-5-yl]-2-phenylacetamide
N-[3-(1H-Indol-2-yl)-1H-thieno[2,3-c]pyrazol-5-yl]benzenesulphonamide
2-Methanesulphonyl-N-[3-(5-methoxy-1H-indol-2-yl)-1H-thieno[2,3-c]pyrazol-5-yl]benzenesulphonamide
N-[3-(5-Methoxy-1H-indol-2-yl)-1H-thieno[2,3-c]pyrazol-5-yl]benzenesulphonamide
N-[3-(1H-Pyrrol-2-yl)-1H-thieno[2,3-c]pyrazol-5-yl]benzenesulphonamide
N-[3-(1H-Imidazol-2-yl)-1H-thieno[2,3-c]pyrazol-5-yl]benzenesulphonamide
N-(3-Benzofuran-2-yl-1H-thieno[2,3-c]pyrazol-5-yl)benzenesulphonamide
N-(3-Benzo[b]thiophen-2-yl-1H-thieno[2,3-c]pyrazol-5-yl)benzenesulphonamide
N-[3-((E)-Styryl)-1H-thieno[2,3-c]pyrazol-5-yl]benzenesulphonamide
2-Phenyl-N-(3-phenyl-1H-thieno[2,3-c]pyrazol-5-yl)acetamide

The method for preparing the compounds according to the invention can be represented schematically in the following way:

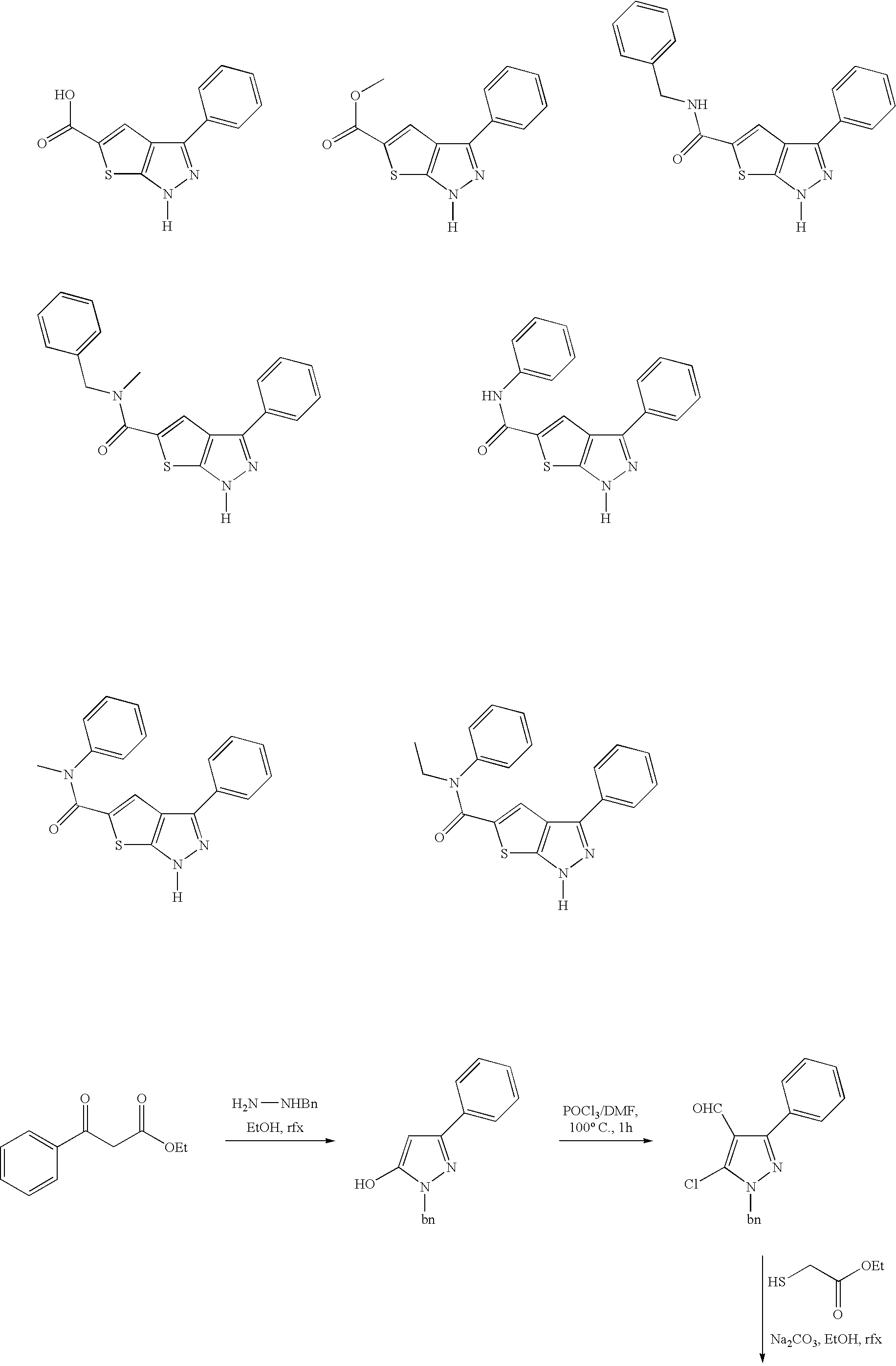

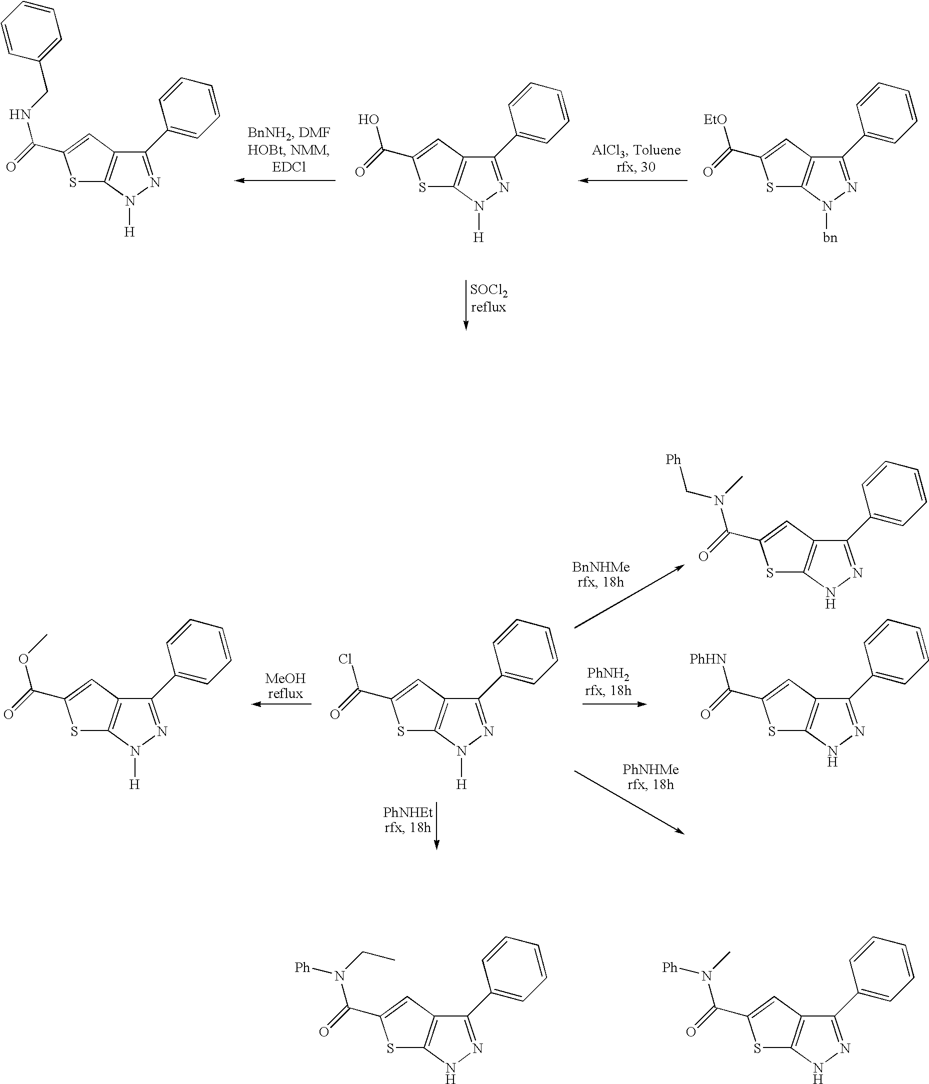

The compounds according to the invention can be used in human therapy and more particularly in the treatment of cancer, more particularly cancers sensitive to Aurora-2 or KOR inhibitors. They are also used to treat Alzheimer's disease and more particularly on individuals sensitive to GSK-3β inhibitors.

The present invention will be described more thoroughly using the following examples which should not be considered as limiting the invention.

### EXAMPLE 1

### Preparation of 3-phenyl-1H-thieno[2,3-c]pyrazole-5-carboxylic acid

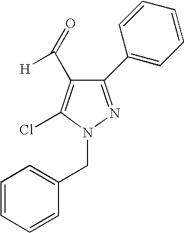

1-Benzyl-5-chloro-3-phenyl-1H-pyrazole-4 carboxaldehyde is prepared according to Malhotra et al., J. Het. Chem., 1991, 28(8), 1837. 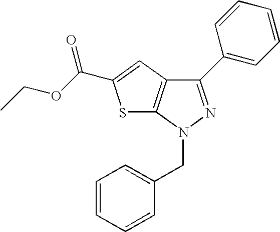
The ethyl ester of 1-benzyl-3-phenyl-1H-thieno[2,3-c]pyrazole-5-carboxylic acid is prepared in the following way:

A suspension containing 2.6 g (8.761 mmol) of 1-benzyl-5-chloro-3-phenyl-1H-pyrazole-4 carboxaldehyde, 1 cm³ (8.761 mmol) of ethyl thio-glycolate and 1.55 g (14.63 mmol) of sodium carbonate in 90 cm³ of ethanol is brought to a temperature in the region of the reflux temperature for 2 hours. The suspension is filtered while boiling through a sintered glass funnel, the solid is rinsed with 2 times 10 cm³ of boiling ethanol, and the filtrate is returned to a temperature in the region of 20° C., before being poured into 200 cm³ of isopropyl oxide. The white precipitate thus obtained is filtered off through a sintered glass funnel, washed with 4 times 25 cm³ of isopropyl oxide and dried in a desiccator under vacuum. 1.8 g of the ethyl ester of 1-benzyl-3-phenyl-1H-thieno[2,3-c]pyrazole-5-carboxylic acid are thus obtained in the form of a white solid which melts at 142° C.

Mass spectrum (EI): m/z=362 M^{+.} (base peak); m/z=289 (M−C₃H₅O₂)⁺; m/z=91 C₇H₇⁺ 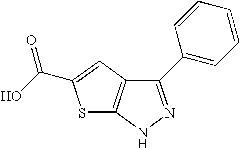
The 3-phenyl-1H-thieno[2,3-c]pyrazole-5-carboxylic acid is prepared in the following way:

A suspension containing 1.1 g (8.28 mmol) of aluminium trichloride in 45 cm³ of toluene is stirred under an inert atmosphere, and then 1.5 g (4.14 mmol) of the ethyl ester of 1-benzyl-3-phenyl-1H-thieno[2,3-c]pyrazole-5-carboxylic acid are added, and the resulting suspension is heated at a temperature in the region of the reflux temperature for 40 minutes. The reaction medium is then cooled to a temperature in the region of 20° C., and then poured over 100 g of ice. The solid is filtered off through a sintered glass funnel, and then rinsed with 3 times 30 cm³ of water. The filtrate is extracted with 3 times 150 cm³ of ethyl acetate, and the organic phases are pooled, washed with 3 times 50 cm³ of water, separated after settling out, dried over anhydrous MgSO₄ and then filtered through paper. After evaporation of the solvent, the residue thus obtained is mixed with the solid previously isolated, to give 1.6 g of a yellow solid. This solid is taken up in 100 cm³ of an aqueous 2N sodium hydroxide solution and stirred vigorously for 30 minutes. The suspension is filtered through Celite, and the Celite is rinsed with 3 times 30 cm³ of 2N NaOH and 3 times 30 cm³ of water. The filtrate is acidified to pH=2 by adding aqueous 5N HCl, taking care to maintain the temperature of the reaction medium below 28° C. The suspension is stirred vigorously for 30 minutes, and the solid is then filtered off through a sintered glass funnel, rinsed with 6 times 50 cm³ of water and dried in a desiccator under vacuum for 18 hours. 0.88 g of 3-phenyl-1H-thieno[2,3-c]pyrazole-5-carboxylic acid is thus obtained, in the form of a white solid having a melting point above 260° C.

Mass spectrum (EI): m/z=244 M^{+.}(base peak); m/z=104 C₇H₆N⁺; m/z=77 C₆H₅^{+IR spectrum as KBr disc:} 3229; 3062; 2926; 2515; 1825; 1678; 1544; 1515; 1455; 1288; 1245; 1184; 1093; 1024; 974; 769; 712; 693 and 601 cm⁻¹ ¹H NMR spectrum (300 MHz, (CD₃)₂SO, δ in ppm): 7.43 (broad t, J=7.5 Hz: 1H); 7.55 (broad t, J=7.5 Hz: 2H); 7.99 (broad d, J=7.5 Hz: 2H); 8.12 (s: 1H).

### EXAMPLE 2

### Preparation of the Methyl Ester of 3-phenyl-1H-thieno[2,3-c]pyrazole-5-carboxylic acid

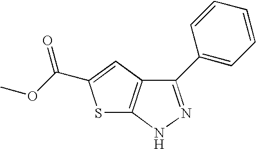 The methyl ester of 3-phenyl-1H-thieno[2,3-c]pyrazole-5-carboxylic acid is prepared in the following way:

A solution containing 0.07 g of 3-phenyl-1H-thieno[2,3-c]pyrazole-5-carboxylic acid in 5 cm³ of SOCl₂ and 5 cm³ of methanol is heated at a temperature in the region of the reflux temperature for 1 hour. The pale yellow solution is concentrated to dryness under reduced pressure, and the residue thus obtained is purified by chromatography on a column 1.5 cm in diameter containing 21 cm of 70-200 μm silica, at atmospheric pressure, using as eluent pure dichloromethane and then a dichloromethane/ethyl acetate mixture (95/5 by volume). The fractions containing the expected product are pooled and concentrated under reduced pressure, to give 0.027 g of the methyl ester of 3-phenyl-1H-thieno[2,3-c]pyrazole-5-carboxylic acid, in the form of an off-white solid.

Mass spectrum (EI): m/z=258 M^{+.}(base peak); m/z=227 (M−OMe)⁺; m/z=104 C₇H₆N⁺; m/z=77 C₆H₅⁺IR spectrum as KBr disc: 3268; 3064; 2954; 1682; 1516; 1483; 1447; 1431; 1320; 1261; 1109; 1058; 1023; 971; 803; 767; 760; 712; 695; 684 and 595 cm⁻¹ ¹H NMR spectrum (300 MHz, (CD₃)₂SO, δ in ppm): 3.90 (s: 3H); 7.42 (broad t, J=7.5 Hz: 1H); 7.54 (broad t, J=7.5 Hz: 2H); 8.00 (broad d, J=7.5 Hz: 2H); 8.24 (s: 1H); from 13.30 to 14.20 (broad unresolved peak:1H).

### EXAMPLE 3

### Preparation of 3-phenyl-1H-thieno[2,3-c]pyrazole-5-carboxylic acid benzylamide

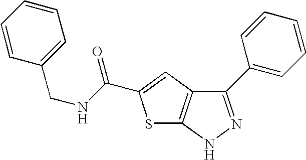 The 3-phenyl-1H-thieno[2,3-c]pyrazole-5-carboxylic acid benzylamide is prepared in the following way:

A solution containing 0.122 g (0.5 mmol) of 3-phenyl-1H-thieno[2,3-c]pyrazole-5-carboxylic acid in 10 cm³ of DMF is stirred at a temperature in the region of 20° C. 0.007 g (0.05 mmol) of 1-hydroxybenzotriazole, 0.056 cm³ (0.5 mmol) of benzylamine and 0.061 cm³ (0.55 mmol) of N-methylmorpholine are then added, and the solution thus obtained is stirred for 1 hour at a temperature in the region of 20° C. 0.108 g (0.55 mmol) of 1-(3-dimethylaminopropyl)-3-ethylcarbodiimide hydrochloride is then added, and the yellow solution thus obtained is stirred for 18 hours at a temperature in the region of 20° C. The reaction medium is then poured over 120 cm³ of water, the aqueous phase is extracted with 3 times 50 cm³ of ethyl acetate, and the organic phases are pooled, washed with 2 times 50 cm³ of water, separated after settling out, dried over anhydrous MgSO₄, then filtered through paper, and then concentrated under reduced pressure. The residue thus obtained is purified by chromatography on a column 1.5 cm in diameter containing 21 cm of 70-200 μm silica, at atmospheric pressure, using as eluent a dichloromethane/ethyl acetate mixture (75/25 by volume). The fractions containing the expected product are pooled and concentrated under reduced pressure, to give 0.097 g of a white solid, which is washed with 3 times 10 cm³ of ethyl ether and dried under vacuum. The solid thus obtained is washed with 3 times 20 cm³ of isopropyl ether and dried under vacuum, to give 0.097 g of 3-phenyl-1H-thieno[2,3-c]pyrazole-5-carboxylic acid benzylamide in the form of a white solid which melts at between 98 and 101° C.

Mass spectrum (EI): m/z=333 M^{+.}(base peak); m/z=227 (M−C₇H₈N)⁺; m/z=106 C₇H₈N⁺; m/z=104 C₇H₆N⁺; m/z=77 C₆H₅^{+IR spectrum as KBr disc:} 3413; 3062; 3032; 292 ¹H NMR spectrum (300 MHz, (CD₃)₂SO, δ in ppm): 4.52 (d, J=6 Hz: 2H); from 7.20 to 7.40 (mt: 5H); 7.42 (broad t, J=7.5 Hz: 1H); 7.55 (broad t, J=7.5 Hz: 2H); 7.93 (broad d, J=7.5 Hz: 2H); 8.30 (s: 1H); 9.09 (t, J=6 Hz: 1H); from 13.50 to 14.20 (broad unresolved peak: 1H).6; 1625; 1546; 1494; 1454; 1294; 1255; 975; 767; 711; 698 and 600 cm⁻¹

### EXAMPLE 4

### Preparation of 3-phenyl-1H-thieno[2,3-c]pyrazole-5-carboxylic acid N-methylbenzylamide

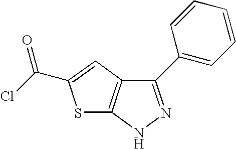 The acid chloride of 3-phenyl-1H-thieno[2,3-c]pyrazole-5-carboxylic acid can be prepared in the following way:

A supension containing 0.244 g (1 mmol) of 3-phenyl-1H-thieno[2,3-c]pyrazole-5-carboxylic acid in 2.2 cm³ of thionyl chloride is brought, with stirring, to a temperature in the region of the reflux temperature, and maintained at this temperature for 18 hours. The suspension is then returned to a temperature in the region of 20° C., and then diluted with 10 cm³ of chloroform. The resulting suspension is filtered through a sintered glass funnel and the solid is washed with 3 times 10 cm³ of chloroform and then dried under vacuum, to give 0.224 g of the acid chloride of 3-phenyl-1H-thieno[2,3-c]pyrazole-5-carboxylic acid, used as it stands for the following reaction: 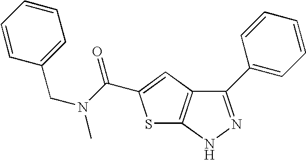
The 3-phenyl-1H-thieno[2,3-c]pyrazole-5-carboxylic acid N-methylbenzylamide can be prepared in the following way:

The acid chloride of 3-phenyl-1H-thieno[2,3-c]pyrazole-5-carboxylic acid (0.111 g, 0.423 mmol) prepared as above is suspended in 5.5 cm³ of N-methylbenzylamine (42.3 mmol) and the resulting suspension is heated, with stirring and under an inert atmosphere, to a temperature in the region of 60° C. After heating for 18 hours, the reaction medium is returned to a temperature in the region of 20° C. and then poured over 100 cm³ of aqueous 2N HCl. The yellow suspension thus obtained is filtered through a sintered glass funnel, and the solid is washed with 3 times 25 cm³ of water and then with 3 times 25 cm³ of ethyl acetate. The filtrate is separated after settling out, the aqueous phase is extracted with 3 times 50 cm³ of ethyl acetate, and the organic phases are pooled and then dried over anhydrous MgSO₄, filtered through paper and then concentrated to dryness under reduced pressure. The residue is purified by chromatography on a column 2 cm in diameter containing 15 cm of 20-45 μm silica, at atmospheric pressure, using as eluent a dichloromethane/ethyl acetate mixture (75/25 by volume). The fractions containing the expected product are pooled and concentrated under reduced pressure, to give 0.024 g of a yellow foam. This product is purified by LC/MS using a Waters FractionLynx system composed of a Waters model 600 gradient pump, a Waters model 515 regeneration pump, a Waters Reagent Manager dilution pump, a Waters model 2700 auto-injector, two Rheodyne model LabPro valves, a Waters model 996 diode array detector, a Waters model ZMD mass spectrometer and a Gilson model 204 fraction collector. The system was monitored by the Waters FractionLynx software. The separation was performed alternately on two Waters Symmetry columns (C₁₈, 5 μM, 19×50 mm, catalogue reference 186000210), one column undergoing regeneration with a 95/5 (v/v) water/acetonitrile mixture containing 0.07% (v/v) of trifluoroacetic acid, while the other column was being used for separation. The columns were eluted using a linear gradient of 5 to 95% of acetonitrile containing 0.07% (v/v) of trifluoroacetic acid in water containing 0.07% (v/v) of trifluroacetic acid, at a flow rate of 10 mL/min. At the outlet of the separation column, one-thousandth of the effluent is separated by means of an LC Packing Accurate, diluted with methyl alcohol, at a flow rate of 0.5 mL/min, and sent to the detectors, in a proportion of 75% to the diode array detector, and the remaining 25% to the mass spectrometer. The rest of the effluent (999/1000) is sent to the fraction collector, where the flow is discarded as long as the mass of the expected product is not detected by the FractionLynx software. The molecular formulae of the expected products are supplied to the FractionLynx software, which actuates the collection of the product when the mass signal detected corresponds to the ion [M+H]⁺ and/or to [M+Na]⁺. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, the collection is also actuated when the mass signal for the ion [M+2H]⁺⁺ and/or [M+Na+H]⁺⁺ are detected. The products were collected in a tared glass tube. After collection, the solvents were evaporated off in a Savant AES 2000 or Genevac HT8 centrifuge evaporator and the product masses were determined by weighing the tubes after evaporating off the solvents. 0.012 g of 3-phenyl-1H-thieno[2,3-c]pyrazole-5-carboxylic acid N-methylbenzylamide is thus recovered, in the form of a white solid.

LC/MS analysis: The LC/MS analyses were performed on a Micromass Model LCT device connected to an HP 1100 device. The abundance of the products was measured using an HP G1315A diode array detector over a wavelength range of 200-600 nm and a Sedex 65 light scattering detector. The mass spectra were acquired over a range of 180 to 800. The data were analyzed using the Micromass MassLynx software. The separation was performed on a Hypersil BDS C18, 3 μm (50×4.6 mm) column, eluting with a linear gradient of 5 to 90% of acetonitrile containing 0.05% (v/v) of trifluoroacetic acid (TFA) in water containing 0.05% (v/v) TFA, over 3.5 minutes at a flow rate of 1 mL/min. The total analysis time, including the period for re-equilibrating the column, is 7 minutes.

LC/MS peak: [M+H]⁺=348 Retention time=3.63 minutes.

### EXAMPLE 5

### Method for Preparing 3-phenyl-1H-thieno[2,3-c]pyrazole-5-carboxylic acid Phenylamide

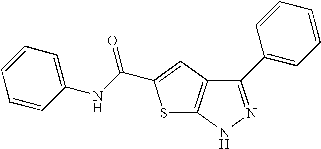 The 3-phenyl-1H-thieno[2,3-c]pyrazole-5-carboxylic acid phenylamide can be prepared in the following way:

The acid chloride of 3-phenyl-1H-thieno[2,3-c]pyrazole-5-carboxylic acid (0.111 g, 0.423 mmol) prepared as above is suspended in 3.9 cm³ of aniline (42.3 mmol), and the resulting suspension is heated, with stirring and under an inert atmosphere, at a temperature in the region of 60° C. After heating for 18 hours, the reaction medium is returned to a temperature in the region of 20° C., and then poured over 100 cm³ of aqueous 2N HCl. The yellow suspension thus obtained is filtered through a sintered glass funnel, and the solid is washed with 3 times 25 cm³ of water and then with 3 times 25 cm³ of ethyl acetate. The filtrate is separated after settling out, the aqueous phase is extracted with 3 times 50 cm³ of ethyl acetate, and the organic phases are pooled, then dried over anhydrous MgSO₄, filtered through paper, and then concentrated to dryness under reduced pressure. The residue thus obtained is purified by chromatography on a column 1.5 cm in diameter containing 20 cm of 20-45 μm silica, at atmospheric pressure, using as eluent a dichloromethane/ethyl acetate mixture (75/25 by volume). The fractions containing the expected product are pooled and concentrated under reduced pressure, to give 0.016 g of 3-phenyl-1H-thieno[2,3-c]pyrazole-5-carboxylic acid phenylamide, in the form of a yellow foam.

Mass spectrum (EI): m/z 319 (M^{+.}); m/z 227 (base peak).

### EXAMPLE 7

### Method for Preparing 3-phenyl-1H-thieno[2,3-c]pyrazole-5-carboxylic acid N-methylphenylamide

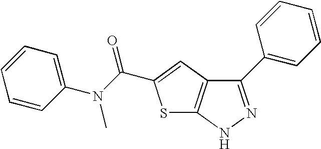 The 3-phenyl-1H-thieno[2,3-c]pyrazole-5-carboxylic acid N-methylphenylamide can be prepared in the following way:

0.067 ml (0.6 mmol) of N-methylaniline, and then 0.085 ml (0.6 mmol) of triethylamine, are added successively, and with stirring, to a suspension containing 0.131 g (0.5 mmol) of acid chloride of 3-phenyl-1H-thieno[2,3-c]pyrazole-5-carboxylic acid prepared as above, in 25 cm³ of chloroform, and the resulting suspension is heated at a temperature in the region of the reflux temperature, and maintained at this temperature for 18 hours. The reaction medium is returned to a temperature in the region of 20° C. and then diluted with 100 cm³ of chloroform and with 100 cm³ of water. The organic phase is separated after settling out, the aqueous phase is extracted with 3 times 50 cm³ of chloroform, and the organic phases are pooled, washed with 3 times 50 cm³ of water, dried over anhydrous MgSO₄, filtered, and then concentrated to dryness under reduced pressure. The residue thus obtained is purified by chromatography on a column 2 cm in diameter containing 24 cm of 70-200 μm silica, at atmospheric pressure, using as eluent a dichloromethane/ethyl acetate mixture (75/25 by volume). The fractions containing the expected product are pooled and concentrated under reduced pressure, to give 0.014 g of 3-phenyl-1H-thieno[2,3-c]pyrazole-5-carboxylic acid N-methylphenylamide, in the form of a yellow foam.

LC/MS analysis: The LC/MS analyses were performed on a Micromass Model LCT device connected to an HP 1100 device. The abundance of the products was measured using an HP G1315A diode array detector over a wavelength range of 200-600 nm and a Sedex 65 light scattering detector. The mass spectra were acquired over a range of 180 to 800. The data were analyzed using the Micromass MassLynx software. The separation was performed on a Hypersil BDS C18, 3 μm (50×4.6 mm) column, eluting with a linear gradient of 5 to 90% of acetonitrile containing 0.05% (v/v) of trifluoroacetic acid (TFA) in water containing 0.05% (v/v) TFA, over 3.5 minutes at a flow rate of 1 mL/min. The total analysis time, including the period for re-equilibrating the column, is 7 minutes.

LC/MS peak: [M+H]⁺=334 Retention time=3.57 minutes.

### EXAMPLE 8

### Preparation of 3-phenyl-1H-thieno[2,3-c]pyrazole-5-carboxylic acid N-ethylphenylamide

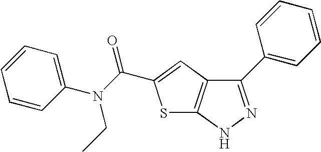 The 3-phenyl-1H-thieno[2,3-c]pyrazole-5-carboxylic acid N-ethylphenylamide can be prepared in the following way:

0.078 ml (0.6 mmol) of N-ethylaniline, then 0.085 ml (0.6 mmol) of triethylamine, are added successively, and with stirring, to a suspension containing 0.131 g (0.5 mmol) of acid chloride of 3-phenyl-1H-thieno[2,3-c]pyrazole-5-carboxylic acid prepared as above, in 25 cm³ of chloroform, and the resulting suspension is heated to a temperature in the region of the reflux temperature, and maintained at this temperature for 18 hours. The reaction medium is returned to a temperature in the region of 20° C., and then diluted with 100 cm³ of chloroform and with 100 cm³ of water. The organic phase is separated after settling out, the aqueous phase is extracted with 3 times 50 cm³ of chloroform, and the organic phases are pooled, washed with 3 times 50 cm³ of water, dried over anhydrous MgSO₄, filtered, and then concentrated to dryness under reduced pressure. The residue thus obtained is purified by chromatography on a column 2 cm in diameter containing 24 cm of 70-200 μm silica, at atmospheric pressure, using as eluent a dichloromethane/ethyl acetate mixture (75/25 by volume). The fractions containing the expected product are pooled and concentrated under reduced pressure, to give 0.010 g of 3-phenyl-1H-thieno[2,3-c]pyrazole-5-carboxylic acid N-ethylphenylamide, in the form of a yellow foam.

LC/MS analysis: The LC/MS analyses were performed on a Micromass Model LCT device connected to an HP 1100 device. The abundance of the products was measured using an HP G1315A diode array detector over a wavelength range of 200-600 nm and a Sedex 65 light scattering detector. The mass spectra were acquired over a range of 180 to 800. The data were analyzed using the Micromass MassLynx software. The separation was performed on a Hypersil BDS C18, 3 μm (50×4.6 mm) column, eluting with a linear gradient of 5 to 90% of acetonitrile containing 0.05% (v/v) of trifluoroacetic acid (TFA) in water containing 0.05% (v/v) TFA, over 3.5 minutes at a flow rate of 1 mL/min. The total analysis time, including the period for re-equilibrating the column, is 7 minutes.

LC/MS peak: [M+H]⁺=348 Retention time=3.77 minutes.

### EXAMPLE 9:

### Preparation of 3-((E)-Styryl)-1H-thieno[2,3-c]pyrazole-5-carboxylic acid phenoxyamide

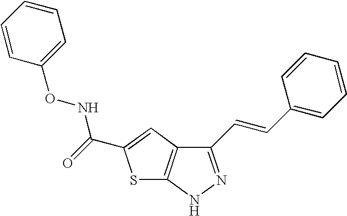 3-((E)-Styryl)-1H-thieno[2,3-c]pyrazole-5-carboxylic acid phenoxyamide is prepared in the following way:

1.5 cm³ (3.00 mmol, 15 equivalents) of aqueous 2N HCl are added to a solution stirred under argon and containing 0.083 g (0.191 mmol) of 1-(1-ethoxyethyl)-3-((E)-styryl)-1H-thieno[2,3-c]pyrazole-5-carboxylic acid phenoxyamide in 5 cm³ of tetrahydrofuran, and the solution thus obtained is stirred at a temperature in the region of 20° C. for 72 hours. The reaction medium is then concentrated under vacuum at reduced pressure, and the residue thus obtained is purified by preparative LC-MS using a Waters FractionsLynx system composed of a Waters model 600 gradient pump, a Waters model 515 regeneration pump, a Waters Reagent Manager dilution pump, a Waters model 2700 auto-injector, two Rheodyne model LabPro valves, a Waters model 996 diode array detector, a Waters model ZMD mass spectrometer and a Gilson model 204 fraction collector. The system was controlled by the Waters FractionLynx software. Separation was carried out alternately on two Waters Symmetry columns (C₁₈, 5 μM, 19×50 mm, catalogue reference 186000210), one column undergoing regeneration with a 95/5 (v/v) water/acetonitrile mixture comprising 0.07% (v/v) of trifluoroacetic acid, while the other column was being used for separation. The columns were eluted using a linear gradient from 5 to 95% of acetonitrile comprising 0.07% (v/v) of trifluoroacetic acid in water comprising 0.07% (v/v) of trifluroacetic acid, at a flow rate of 10 mL/min. At the outlet of the separation column, one-thousandth of the effluent is separated by means of an LC Packing Accurate, diluted with methyl alcohol, at a flow rate of 0.5 mL/min and sent to the detectors, in a proportion of 75% to the diode array detector, and the remaining 25% to the mass spectrometer. The rest of the effluent (999/1000) is sent to the fraction collector, where the flow is discarded for as long as the mass of the expected product is not detected by the FractionLynx software. The molecular formulae of the expected products are supplied to the FractionLynx software, which actuates the collection of the product when the mass signal detected corresponds to the ion [M+H]⁺ and/or to [M+Na]⁺. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, the collection is also actuated when the mass signal for the ion [M+2H]⁺⁺ and/or [M+Na+H]⁺⁺ is detected. 0.030 g of 3-((E)-styryl-1H-thieno[2,3-c]pyrazole-5-carboxylic acid phenoxyamide is thus recovered in the form of a white solid.

Mass spectrum (EI): m/z 361 [M^{+.}], m/z 94 (base peak): Ph-O⁺IR spectrum (as KBr disc): 3413; 3193; 3058; 2929; 1646; 1591; 1533; 1489; 1287; 1195; 1160; 1097; 954; 752; 689; 594 and 504 cm⁻¹ ¹H NMR spectrum (400 MHz)—δ in ppm—in d6-DMSO: 7.04 (broad t, J=8.0 Hz, 1H); 7.14 (broad d, J=8.0 Hz, 2H); from 7.28 to 7.39 (unresolved peak, 5H); 7.43 (broad t, J=8.0 Hz, 2H); 7.66 (broad d, J=8.0 Hz, 2H); 8.16 (broad unresolved peak, 1H); 12.5 (broad unresolved peak, 1H); 13.8 (broad unresolved peak, 1H). 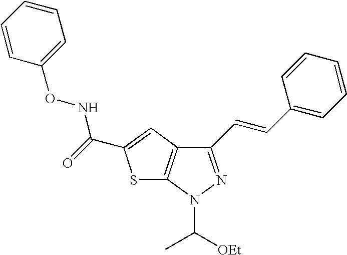
1-(1-Ethoxyethyl)-3-((E)-styryl-1H-thieno[2,3-c]pyrazole-5-carboxylic acid phenoxyamide is prepared in the following way:

A solution containing 0.200 g (0.584 mmol) of 1-(1-ethoxyethyl)-3-((E)-styryl)-1H-thieno[2,3-c]pyrazole-5-carboxylic acid in 10 cm³ of acetonitrile is stirred under argon at ambient temperature. The following are then added successively: 0.118 g (1.168 mmol, 2 equivalents) of triethylamine, 0.0893 g (0.613 mmol, 1.05 equivalent) of O-phenylhydroxylamine hydrochloride, and then 0.197 g (0.613 mmol, 1.05 equivalent) of O-(1H-benzotriazol-1-yl)-N,N,N′,N′-tetramethyluronium tetrafluoroborate (TBTU). The reaction medium is stirred at a temperature in the region of 20° C. for 18 hours and is then heated to a temperature in the region of 80° C. for 3 hours 30 min. The reaction medium is returned to a temperature in the region of 20° C., and poured into a mixture containing 40 cm³ of brine and 40 cm³ of EtOAc. The organic phase is separated by settling out, the aqueous phase is extracted with 2 times 40 cm³ of EtOAc, and the organic phases are combined, washed with 50 cm³ of 5N HCl, then with 50 cm³ of water, then with 40 cm³ of a saturated aqueous NaHCO₃ solution, then with 50 cm³ of water, and then dried over anhydrous MgSO₄, filtered through paper, and then concentrated to dryness under reduced pressure. The residue thus obtained is purified by flash chromatography on a Puriflash cartridge containing 40 g of SiO₂ (20 μm, spherical), elution being carried out with a cyclohexane/EtOAc mixture (75/25 by volume) at a flow rate of 10 ml/min. 0.094 g of 1-(1-ethoxyethyl)-3-((E)-styryl)-1H-thieno[2,3-c]pyrazole-5-carboxylic acid phenoxyamide is thus recovered in the form of a red solid.

Mass spectrum: EI: m/z 433 [M^{+.}], m/z 339: 433-PhO m/z 267 (base peak): 339-C₂H₅—OCH—CH₃ m/z 94: PhO⁺ 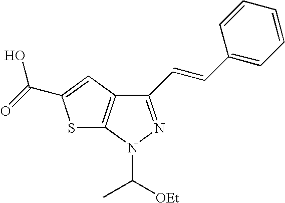
1-(1-Ethoxyethyl)-3-((E)-styryl-1H-thieno[2,3-c]pyrazole-5-carboxylic acid is prepared in the following way:

A solution containing 1 g (2.7 mmol) of 1-(1-ethoxyethyl)-3-((E)-styryl)-1H-thieno[2,3-c]pyrazole-5-carboxylic acid ethyl ester (prepared as above) in 8 cm³ of tetrahydrofuran is treated successively, with stirring, with 1 cm³ of ethanol, 1 cm³ of water and 0.300 g (5.4 mmol, 2 equivalents) of KOH. The reaction medium is heated to a temperature in the region of 85° C. After 3 hours, the heating is stopped, and the reaction medium is returned to a temperature in the region of 20° C. and concentrated under vacuum. The residue is dissolved in 75 cm³ of water, and extracted with 2 times 75 cm³ of ethyl ether. The aqueous phase is acidified to a pH of approximately 5 by adding solid citric acid, and is then extracted with 3 times 50 cm³ of EtOAc. The organic phases are combined, dried over anhydrous MgSO₄, filtered through paper, and then concentrated under reduced pressure. 0.850 g of 1-(1-ethoxyethyl)-3-((E)-styryl)-1H-thieno[2,3-c]pyrazole-5-carboxylic acid is thus recovered in the form of a yellow solid.

Mp=160° C. Mass spectrum (EI): m/z 342 [M^{+.}], 270 (base peak): [M^{+.}] —C₂H₅—O—CH—CH₃.

### EXAMPLE 10

### Preparation of 3-[(thiophene-2-carbonyl)amino]-1H-thieno[2,3-c]pyrazole-5-carboxylic acid (1-methyl-1-phenylethyl)amide

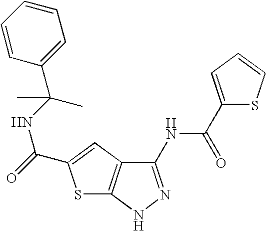 3-[(Thiophene-2-carbonyl)amino-1H-thieno[2,3-c]pyrazole-5-carboxylic acid (1-methyl-1-phenylethyl)amide is prepared in the following way:

0.930 cm³ (1.86 mmol, 15 equivalents) of aqueous 2N HCl are added to a solution stirred under argon and containing 0.060 g (0.124 mmol) of 1-(1-ethoxyethyl)-3-[(thiophene-2-carbonyl)amino]-1H-thieno[2,3-c]pyrazole-5-carboxylic acid (1-methyl-1-phenylethyl)amide in 3 cm³ of tetrahydrofuran, and the solution thus obtained is stirred at a temperature in the region of 20° C. for 18 hours. The reaction medium is then concentrated under vacuum at reduced pressure, and the residue thus obtained is purified by preparative LC-MS using a Waters FractionsLynx system composed of a Waters model 600 gradient pump, a Waters model 515 regeneration pump, a Waters Reagent Manager dilution pump, a Waters model 2700 auto-injector, two Rheodyne model LabPro valves, a Waters model 996 diode array detector, a Waters model ZMD mass spectrometer and a Gilson model 204 fraction collector. The system was controlled by the Waters FractionLynx software. Separation was carried out alternately on two Waters Symmetry columns (C₁₈, 5 μM, μ19×50 mm, catalogue reference 186000210), one column undergoing regeneration with a 95/5 (v/v) water/acetonitrile mixture comprising 0.07% (v/v) of trifluoroacetic acid, while the other column was being used for separation. The columns were eluted using a linear gradient from 5 to 95% of acetonitrile comprising 0.07% (v/v) of trifluoroacetic acid in water comprising 0.07% (v/v) of trifluroacetic acid, at a flow rate of 10 mL/min. At the outlet of the separation column, one-thousandth of the effluent is separated by means of an LC Packing Accurate, diluted with methyl alcohol, at a flow rate of 0.5 mL/min and sent to the detectors, in a proportion of 75% to the diode array detector, and the remaining 25% to the mass spectrometer. The rest of the effluent (999/1000) is sent to the fraction collector, where the flow is discarded for as long as the mass of the expected product is not detected by the FractionLynx software. The molecular formulae of the expected products are supplied to the FractionLynx software, which actuates the collection of the product when the mass signal detected corresponds to the ion [M+H]⁺ and/or to [M+Na]⁺. In certain cases, depending on the analytical LC/MS results, when an intense ion corresponding to [M+2H]⁺⁺ was detected, the value corresponding to half the calculated molecular mass (MW/2) is also supplied to the FractionLynx software. Under these conditions, the collection is also actuated when the mass signal for the ion [M+2H]⁺⁺ and/or [M+Na+H]⁺⁺ is detected. 0.013 g of 3-[(thiophene-2-carbonyl)amino]-1H-thieno[2,3-c]pyrazole-5-carboxylic acid (1-methyl-1-phenylethyl)amide is thus recovered in the form of a white solid.

Mass spectrum (EI): m/z 410: [M^{+.}], m/z 111 (base peak): thiophene-CO⁺, m/z 395: [M^{+.}]-CH₃ m/z 276: [M^{+.}]-PhC(CH₃)₂—NH IR spectrum (as KBr disc): 3414; 2976; 1642; 1536; 1448; 1416; 1289; 1251; 1169; 738; 719; 700 and 557 cm⁻¹ ¹H NMR spectrum (300 MHz)—δ in ppm—in d6-DMSO: 1.67 (s, 6H); 7.17 (broad t, J=7.5 Hz, 1H); 7.23 (broad t, J=4.5 Hz, 1H); 7.29 (broad t, J=7.5 Hz, 2H); 7.37 (broad d, J=7.5 Hz, 2H); 7.88 (broad d, J=4.5 Hz, 1H); 8.15 (broads, 1H); 8.19 (s, 1H); 8.68 (broads, 1H); 11.2 (broad unresolved peak, 1H); 12.8 (broad unresolved peak, 1H) 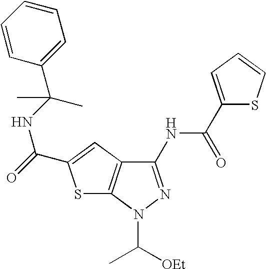
1-(1-Ethoxyethyl)-3-[(thiophene-2-carbonyl)amino]-1H-thieno[2,3-c]pyrazole-5-carboxylic acid (1-methyl-1-phenylethyl)amide is prepared in the following way:

0.670 ml (1.34 mmol, 4.4 equivalents) of a 2M solution of AlMe₃ in toluene is added, dropwise, over a period of around 10 minutes, to a solution stirred under argon and containing 0.181 g (1.34 mmol, 4.4 equivalents) of (1-methyl-1-phenylethyl)amine (cumylamine) in 2.5 cm³ of toluene. The reaction medium thus obtained is stirred for 45 minutes, and then a solution containing 0.120 g (0.3 mmol) of 1-(1-ethoxyethyl)-3-[(thiophene-2-carbonyl)-amino]-1H-thieno[2,3-c]pyrazole-5-carboxylic acid ethyl ester in 2.5 cm³ of toluene is added dropwise over a period of approximately 5 minutes. The solution thus obtained is heated at a temperature close to reflux for 17 hours. The reaction medium is then returned to a temperature in the region of 20° C., and is then diluted with 10 cm³ of aqueous 1N HCl. The organic phase is separated by settling out, the aqueous phase is extracted with 3 times 15 cm³ of EtOAc, and the organic phases are combined, washed with 2 times 10 cm³ of brine, dried over anhydrous MgSO₄ and then filtered through paper and concentrated to dryness under reduced pressure. The residue thus obtained is purified by chromatography at atmospheric pressure on a column 1.8 cm in diameter containing 30 cm of SiO₂ (mean particle size of 20-45 mm), elution being carried out with a cyclohexane/EtOAc mixture (7/3 by volume), to give 0.100 g of 1-(1-ethoxyethyl)-3-[(thiophene-2-carbonyl)amino]-1H-thieno-[2,3-c]pyrazole-5-carboxylic acid (1-methyl-1-phenylethyl)amide in the form of a white solid.

Mass spectrum (EI): m/z 482: [M^{+.}], m/z 111 (base peak): thiophene-CO⁺ m/z 410: [M^{+.}]-C₂H₅O—CH—CH₃, m/z 292: 410⁺-PhC(CH₃)₂ 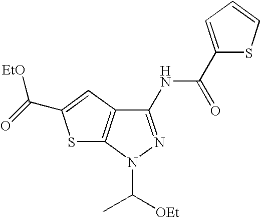
1-(1-Ethoxyethyl)-3-[(thiophene-2-carbonyl)amino]-1H-thieno[2,3-c]pyrazole-5-carboxylic acid ethyl ester is prepared in the following way:

0.220 g (1.729 mmol, 1.2 equivalents) of 2-thiophenecarboxamide, 0.611 g (2.88 mmol, 2 equivalents) of K₃PO₄, 0.027 g (0.144 mmol, 0.1 equivalent) of Cul and 17.3 ml (0.144 mmol, 0.1 equivalent) of trans-1,2-diaminocyclohexane are added, with stirring and under argon, to a solution containing 0.5 g (1.44 mmol) of 3-bromo-1-(1-ethoxyethyl)-1H-thieno[2,3-c]pyrazole-5-carboxylic acid ethyl ester (prepared as above) in 22 cm³ of dioxane. The suspension obtained is heated at a temperature close to reflux for 17 hours. 0.027 g (0.144 mmol, 0.1 equivalent) of Cul and 17.3 ml (0.144 mmol, 0.1 equivalent) of trans-1,2-diaminocyclohexane are then again added, and the suspension thus obtained is heated at a temperature close to reflux for 17 hours. The reaction medium is then returned to a temperature in the region of 20° C., poured into 100 cm³ of water and extracted with 4 times 100 cm³ of EtOAc. The organic phases are combined, washed with 100 cm³ of brine, dried over anhydrous MgSO₄, filtered through paper, and then concentrated to dryness under reduced pressure. The residue thus obtained is purified on a Varian MetaFlash cartridge containing 90 g of SiO₂ of 40 mM, elution being carried out with a cyclohexane/EtOAc mixture (2.5/1 by volume) at a flow rate of 15 ml/min. 0.121 g of 1-(1-ethoxyethyl)-3-[(thiophene-2-carbonyl)amino]-1H-thieno[2,3-c]pyrazole-5-carboxylic acid ethyl ester is thus recovered in the form of a yellow oil.

Mass spectrum (EI): m/z 393: [M^{+.}], m/z 111 (base peak): thiophene-CO⁺ m/z 321: [M^{+.}]-C₂H₅OCH—CH₃

Experimental Protocols Regarding the Biochemical Tests

1. FAK

The inhibitory activity of the compounds on FAK is determined by measuring the inhibition of the autophosphorylation of the enzyme using a time-resolved fluorescence (HTRF) assay.

The complete cDNA of human FAK, the N-terminal end of which was labelled with histidine, was cloned into a baculovirus expression vector pFastBac HTc. The protein was expressed and purified to approximately 70% homogeneity.

The kinase activity is determined by incubating the enzyme (6.6 μg/ml) with various concentrations of test compound in a 50 mM Hepes buffer, pH=7.2, containing 10 mM MgCl₂, 100 μM Na₃VO₄ and 15 μM of ATP for 1 hour at 37° C. The enzyme reaction is stopped by adding Hepes buffer, pH=7.0, containing 0.4 mM KF, 133 mM EDTA and 0.1% BSA, and the labelling is carried out, for 1 to 2 hours at ambient temperature, by adding to this buffer an anti-histidine antibody labelled with XL665 and a monoclonal antibody phosphospecific for tyrosine conjugated to europium cryptate (Eu-K). The characteristics of the two fluorophores are available in G. Mathis et al., Anticancer Research, 1997, 17, pages 3011-3014. The transfer of energy from the excited europium cryptate to the XL665 acceptor is proportional to the degree of autophosphorylation of FAK. The long-lived signal specific for XL-665 is measured in a Packard Discovery plate counter. All the assays are carried out in duplicate and the mean of the two assays is calculated. The inhibition of the FAK autophosphorylation activity with compounds of the invention is expressed as percentage inhibition compared to a control whose activity is measured in the absence of test compound. The [signal at 665 nm/signal at 620 nm] ratio is used to calculate the % inhibition.

2. KDR

The inhibitory effect of the compounds is determined in an assay of substrate phosphorylation by the KDR enzyme in vitro using a scintillation technique (96-well plate, NEN).

The cytoplasmic domain of the human KDR enzyme was cloned in the form of a GST fusion into the baculovirus expression vector pFastBac. The protein was expressed in SF21 cells and purified to approximately 60% homogeneity.

The KDR kinase activity is measured in 20 mM MOPS, 10 mM MgCl2, 10 mM MnCl2, 1 mM DTT, 2.5 mM EGTA, 10 mM β-glycerophosphate, pH=7.2, in the presence of 10 mM MgCl₂, 100 μM Na₃VO₄ and 1 mM NaF. 10 μl of the compound are added to 70 μl of kinase buffer containing 100 ng of KDR enzyme at 4° C. The reaction is triggered by adding 20 μl of solution containing 2 μg of substrate (SH2-SH3 fragment of PLCγ expressed in the form of a GST fusion protein), 2 μCi γ ³³P[ATP] and 2 μM cold ATP. After incubation for 1 hour at 37° C., the reacton is stopped by adding 1 volume (100 μl) of 200 mM EDTA. The incubation buffer is removed and the wells are washed three times with 300 μl of PBS. The radioactivity is measured in each well, using a Top Count NXT ratioactivity counter (Packard).

The background noise is determined by measuring the radioactivity in four different wells containing the radioactive ATP and the substrate alone.

A control of the total activity is measured in four different wells containing all the reagents (γ³³P-[ATP], KDR and PLCγ substrate) but in the absence of compound.

The inhibition of the KDR activity with the compound of the invention is expressed as percentage inhibition of the control activity determined in the absence of compound.

The compound SU5614 (Calbiochem) (1 μM) is included in each plate as an inhibition control.

3. Aurora2

The inhibitory effect of compounds with respect to the Aurora2 kinase is determined with a radioactivity scintillation assay using nickel chelate.

A complete recombinant Aurora2 enzyme, the N-terminal end of which was labelled with histidine, was expressed in E. coli and purified to a quality close to homogeneity.

The C-terminal fragment (Q1687-H2101) of a NUMA (Nuclear protein that associates with the Mitotic Apparatus) expressed in E. coli, and the N-terminal end of which was labelled with histidine, was purified by nickel chelate chromatography and used as substrate in the Aurora2 kinase assay.

In order to determine the kinase activity, the NuMA substrate is equilibrated by chromatography on a Pharmacia PD10 column, in a buffer (50 mM Tris-HCl, pH7.5, 50 mM NaCl, 10 mM MgCl₂) to which 10% (v/v) of glycerol and 0.05% (w/v) of NP40 have been added.

The kinase activity of Aurora2 is measured by scintillation with nickel chelate (New England Nuclear, model SMP107). Each well contains 100 μl of the following solution: 0.02 μM of Aurora2; 0.5 μM of NuMA substrate; 1 μM of ATP to which 0.5 μCi of ATP-[³³P] has been added. The solutions are incubated for 30 minutes at 37° C. The assay buffer is then removed and the wells are rinsed twice with 300 μl of kinase buffer. The radioactivity is measured in each well using a Packard Model Top Count NXT device.

The background noise is deduced from the measurement of radioactivity by measuring, in duplicate, in wells containing the radioactive ATP alone containing buffered kinase treated in the same way as the other samples.

The activity of the control is determined by measuring, in duplicate, the radioactivity in the complete assay mixture (ATP, Aurora2 and the NuMA substrate) in the absence of test compound.

The inhibition of the Aurora2 activity with a compound of the invention is expressed as percentage inhibition of the control activity in the absence of test compound. Staurosporin is added to each plate as an inhibition control.

4. Src

The inhibition of the Src kinase is evaluated by measuring the phosphorylation of the biotinylated cdc2 substrate (Pierce), detected by fluorescence (DELFIA) using an anti-phosphotyrosine antibody labelled with Europium, in 96-well Wallac plates. The c-Src protein used is a recombinant human protein produced in Baculovirus, comprising the SH3 and SH2 domains and the catalytic domain. The enzyme, the substrate and the various concentrations of test compound are placed in the well in a 50 mM Tris buffer containing 10 mM MgCl₂. The reaction is initiated by adding 10 1 μM of ATP. After incubation for 60 minutes at 30° C., the reaction is stopped by adding 75 mM EDTA. 50 μl are taken from each well and transferred into a plate coated with streptavidin. After incubation for 30 minutes at 25° C., the wells are washed with a washing buffer (Wallac) and then the anti-phosphotyrosine antibody (PY20-Europium [Perkin Elmer]) is added in a volume of 75 μl. The plate is incubated for 30 minutes at 25° C. and then an “Enhancer” solution (Wallac) is added before reading the fluorescence using a fluorimeter (Perkin Elmer). The background noise is evaluated in triplicate in wells containing the substrate and the antibody in the absence of enzyme. The activity of the enzyme is measured (in triplicate) in the wells containing all the reagents in the absence of compound. The inhibition of the Src activity is expressed as percentage inhibition of the control activity determined in the absence of compound. The compound PP2 (Calbiochem) is included at various concentrations in each experiment as an inhibition control.

5. Tie2

The coding sequence of human Tie2 corresponding to the amino acids of the intracellular domain 776-1124 was generated by PCR using the cDNA isolated from human placenta as a model. This sequence was introduced into a baculovirus expression vector pFastBacGT in the form of a GST fusion protein.

The inhibitory effect of the molecules is determined in an assay of phosphorylation of PLC by Tie2 in the presence of GST-Tie2 purified to approximately 80% homogeneity. The substrate is composed of the SH2-SH3 fragments of PLC expressed in the form of a GST fusion protein.

The kinase activity of Tie2 is measured in a 20 mM MOPS buffer, pH 7.2, containing 10 mM MgCl₂, 10 mM MnCl₂, 1 mM DTT and 10 mM of glycerophosphate. A reaction mixture composed of 70 μl of kinase buffer containing 100 ng of GST-Tie2 enzyme per well is placed in a FlashPlate 96-well plate kept on ice. 10 μl of the test molecule diluted in DMSO at a maximum concentration of 10% are then added. For a given concentration, each measurement is carried out in quadruplicate. The reaction is initiated by adding 20 μl of a solution containing 2 μg of GST-PLC, 2 μM of cold ATP and 1 μCi of ³³P[ATP]. After incubation for 1 hour at 37° C., the reaction is stopped by adding 1 volume (100 μl) of 200 mM EDTA. After removing the incubation buffer, the wells are washed three times with 300 μl of PBS. The radioactivity is measured on a Wallac MicroBeta 1450.

The inhibition of the Tie2 activity is calculated and expressed as percentage inhibition compared to the control activity determined in the absence of compound.

The products of the examples according to the invention exhibit an activity on the various kinases, and particularly on KDR and Aurora-2, estimated by virtue of the concentration which inhibits 50% of the kinase activity of between 100 nM and 5000 nM.

1. A compound of formula (I): 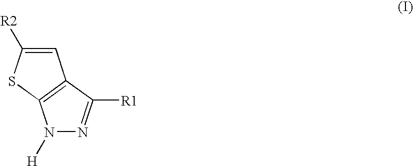
wherein:
R1 is —X—Y
wherein Y is an optionally substituted aryl, heteroaryl, alkyl or cycloalkyl radical
X is a covalent bond, an alkylene, alkenylene, alkynylene, —CO—NR—, —NR—CO—, —SO₂NR—, —NRSO₂—, alkyleneoxy, oxyalkylene, ureido, —NR—O—, —O—NR—, —S—, —SO₂—, —SO—, —O—, —OSO₂—, —NRCOO—, —NRCSNR—, alkylenethio, alkylenesulphone, alkylene sulphoxide, thialkylene, sulphonealkylene or —SO-alkylene;
R2 is same as R1 or halogen, (C₁-C₃)alkyl, (C₂-C₃)alkylene, (C₂-C₃)alkynyl, cycloalkyl or heterocycloalkyl group, or perhaloalkyl, perhaloalkoxy, perhaloalkylthio, hydroxycarbonyl, alkoxycarbonyl, hydroxamate, Ar(CH₂)n(SO₂)NH, Ar(CH₂)n(SO₂)O, Ar(CH₂)n(S)NH, Ar(CH₂)n(S)O, Ar(CH₂)nC(O)O, Ar(CH₂)nC(O)NH, Ar(CH₂)nZC(O), heterocycloalkyl, Alk(CH₂)nZC(O)(CH₂)a, Ar(CH₂)nZC(O)(CH₂)a, Alk(CH₂)a, Ar(CH₂)a, AlkZ(CH₂)a, ArZ(CH₂)a, Alk(CH₂)Z, or Ar(CH₂)Z, wherein Z represents O or NR, Alk is alkyl and Ar is aryl;
wherein R is hydrogen or alkyl, n is 0, 1 or 2 and a is 1 or 2;
wherein all above groups are optionally substituted with alkyl, aryl, amino and/or alkoxy groups; and
with the proviso that, when R2 is R1, then X is not —NH—CO— or —NH—SO₂— or ureido.

2. The compound according to claim 1, wherein Y is a monocyclic or polycyclic heteroaryl group.

3. The compound according to claim 2, wherein Y is a polycyclic heteroaryl group.

4. The compound according to claim 1, wherein X is alkylene, alkenylene, alkynylene, —NR—CO—, —SO₂NR—, —NRSO₂—, alkyleneoxy, oxyalkylene, ureido, —NR—O—, —O—NR—, —O—NR—CO—, —S—, —SO₂—, —SO—, —O—, —OSO₂—, —NRCOO—, —NRCSNR—, alkylenethio, alkylenesulphone, alkylene sulphoxide, thialkylene, sulphonealkylene or —SO-alkylene.

5. The compound according to claim 1, wherein R2 is hydroxamate.

6. The compound according to claim 1, wherein R2 is —CO—NH—C(CH₃)₂—R1, wherein R1 is chosen from aryl, substituted aryl, heteroaryl and substituted heteroaryl.

7. The compound according to claim 1 wherein
R1 is —X—Y
wherein Y is aryl, heteroaryl, alkyl or cycloalkyl,
X is alkylene, alkenylene, alkynylene, NR—CO, SO₂NR, NRSO₂, alkyleneoxy, oxyalkylene or ureido, R2 is same as R1 or halogen, (C₁-C₃)alkyl, (C₂-C₃)alkylene, (C₂-C₃)alkynyl, cycloalkyl or heterocycloalkyl group, or perhaloalkyl, perhaloalkoxy, perhaloalkylthio, hydroxycarbonyl, alkoxycarbonyl, Ar(CH₂)n(SO₂)NH, Ar(CH₂)n(SO₂)O, Ar(CH₂)n(S)NH, Ar(CH₂)n(S)O, Ar(CH₂)nC(O)O, Ar(CH₂)nC(O)NH, Ar(CH₂)nZC(O), heterocycloalkyl, Alk(CH₂)nZC(O)(CH₂)a, Ar(CH₂)nZC(O)(CH₂)a, Alk(CH₂)a, Ar(CH₂)a, AlkZ(CH₂)a, ArZ(CH₂)a, Alk(CH₂)Z, or Ar(CH₂)Z group, wherein Z represents O or NR, Alk is alkyl and Ar is aryl;
wherein R is hydrogen or alkyl, n is 0, 1 or 2 and a is 1 or 2;
wherein all above groups are optionally substituted with alkyl, aryl, amino and/or alkoxy groups
with the proviso that when R2 is R1, then X is not —NH—CO— or —NH—SO₂ or ureido.

8. The compound according to claim 1 , wherein aryl and heteroaryl are selected from the group consisting of phenyl, pyridyl, pyrimidine, triazine, pyrrolyl, imidazolyl, thiazolyl, furyl, thienyl, indolyl, azaindazolyl, isobenzofuranyl, isobenzothienyl, benzoxazolyl, benzothiazolyl, arylvinylene, arylamido, arylcarboxamide, aralkylamine, quinoleyl, isoquinoleyl, cinnolyl, quinazolyl and naphthyridyl.

9. The compound according to claim 8, wherein aryl and heteroaryl are selected from the group consisting of phenyl, pyrrolyl, imidazolyl, thienyl and indolyl.

10. The compound according to claim 9 wherein said heteroaryl is indolyl.

11. The compound according to claim, 1 wherein, X is NR—CO.

12. The compound according to claim 1, wherein R2 is
halogen,
—X—Y wherein X is —CONR— or NR—CO;
Ar(CH₂)n(SO₂)NH,
Ar(CH₂)n(SO₂)O,
Ar(CH₂)nC(O)NH,
or Ar(CH₂)nZC(O) wherein Z represents O or NR, and R3 is hydrogen.

13. A pharmaceutical composition comprising a compound of claim 1 and one or more physiologically acceptable excipients.

14. A method of treating a disease in a patient amenable to modulation of protein kinases selected from the group consisting of KDR, Aurora-2 and GSK-30β, comprising administering to said patient a therapeutically effective amount of a compound according to claim 1.

15. The method according to claim 14 wherein said protein kinase is GSK-3β.

16. The method according to claim 14 wherein said protein kinase is KDR.

17. The method according to claim 14 wherein said protein kinase is Aurora-2.

18. The method according to claim 14 wherein said patient is suffering from cancer caused by the effects of said kinase.

19. The method according to claim 14 wherein said patient is suffering from a neurodegenerative disease.

20. The method according to claim 19 wherein said neurodegenerative disease is caused by the effects of said kinase.
